(19) United States
(12) Patent Application Publication
(10) Pub. No.: US 2019/0333619 A1
(43) Pub. Date: Oct. 31, 2019

(54) Monitoring System for Drug Distribution

(71) Applicant: Bruce H. Levin, Oceanside, NY (US)

(72) Inventor: Bruce H. Levin, Oceanside, NY (US)

(21) Appl. No.: 16/509,288

(22) Filed: Jul. 11, 2019

Related U.S. Application Data
(63) Continuation of application No. 15/081,798, filed on Mar. 25, 2016.
(60) Provisional application No. 62/138,594, filed on Mar. 26, 2015.

(51) Int. Cl. G16H 20/10; G06Q 50/26

(52) U.S. Cl. CPC G16H 20/10; G06Q 50/26

(57) ABSTRACT

A system for tracking one or more drugs comprising a central database adapted to track a drug through its lifecycle from point of manufacture to use by a patient. The system is further adapted to recognize inconsistencies or patterns indicating illicit or improper use of a drug.

### RELATED APPLICATIONS

This application is a continuation of U.S. Ser. No. 15/081798 filed on Mar. 25, 2016, which claims the benefit of U.S. Provisional Application No. 62/138,594 filed Mar. 26, 2015 which are both herein incorporated by reference.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

The present invention relates generally to electromagnetic tag technology and, specifically, to the use of Radio Frequency Identification (RFID) Devices to track proper drug distribution and regimen compliance. In typical settings, once in the possession of a patient, it is difficult to track usage and/or movement of the drugs.

Such drugs may include prescription drugs that are subject to restricted and/or controlled distribution. For example, opioids and other narcotics require strict monitoring for abuse. Tracking usage of a dispensed drug not only ensures that the patient is in possession of the drug, but provides the added benefit of monitoring patient compliance with a prescribed dosing regimen.

Tracking movement of a dispensed drug also enables law enforcement to ascertain legal or illegal possession. Tracking also assists law enforcement in determining the source of the drug.

However, traditional supply chain tracking approaches are insufficient for tracking goods outside the supply chain, such as dispensed drugs. What is needed is a system for tracking restricted-distribution goods that deters diversion by providing for tracking of goods after dispensing to a consumer.

### BRIEF SUMMARY OF THE INVENTION

In some embodiments, systems and methods of the invention are operable for prescription drug tracking as described herein. Drug tracking includes receiving indicia associated with a dispensed prescription and retrieving prescription data associated with the indicia. Tracking also includes retrieving prescription data and determining whether the dispensed prescription was diverted. An indication of whether the dispensed prescription was diverted may also be communicated to a prescriber, pharmacy, manufacturer and/or law enforcement.

In one embodiment, the present invention relates to methods and systems of patient compliance and monitoring as described herein. In other embodiments, patient compliance and monitoring includes receiving patient information, and prescription data associated with the patient information.

A prescriber, pharmacy, manufacturer, treating center, law enforcement, and other verifiable or authorized interested entities and combinations thereof are potential users of the present invention. Compliance monitoring may further include receiving prescription data associated with a dispensed prescription, the DEA Registration number associated with the prescriber, and determining whether the patient is compliant by analyzing prescription data. Compliance monitoring may also include issuing an indication or notification of whether the patient is compliant to the prescriber.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

Systems and methods for drug tracking and/or patient compliance monitoring are described herein. Controlled substances and other drugs are a medical necessity with potential for abuse and misuse, expiration and verifiable disposal and subject to recall. As a result, regulating controlled drugs is a substantial component of federal U.S. drug policy, and encompasses regulation of manufacture, importation, possession, use and distribution of these drugs.

Large numbers of controlled drugs are routinely made available for dispensing via prescriptions and generally include opioids such as codeine, hydrocodone, oxycodone or psychostimulants such as methylphenidate and amphetamine, opioid intermediates, depressants such as some barbiturates, benzodiazepines, narcotics, steroids such as anabolic steroids, and/or the like,

Once a controlled drug enters the supply chain there is a need to maintain the ability to effectively track the drug from the container level to the unit dosage level. Additional challenges include patient compliance, counterfeiting, and diversion.

Accordingly, embodiments of the present invention described herein improve available tracking, compliance and authentication techniques by providing a unified or cohesive monitoring system instead of the disparate systems currently, in use. In a specific embodiment, a central database is used to track a drug through a product's lifecycle from manufacture to use by a patient. At any given point in the lifecycle of a drug, the system of the present invention is adapted to recognize inconsistencies or patterns indicating illicit or improper use.

On feature of the present invention is that it monitors the global quantities of a drug moving through the supply chain to conduct an intelligent analysis. This requires an efficient method to quickly and efficiently track a drug from large quantity shipments to individual or unit dose dispensing. The teachings of U.S. Pat. No. 6,861,954 may be implemented to accomplish this needed feature. The disclosure of this patent is incorporated herein by reference in its entirety.

In one embodiment, a centralized system is configured to monitor a drug as it travels from manufacturer, to distributor, to pharmacy and the prescriber (doctor) as well as the patient. At each step in the lifecycle, the system is programmed with the ability to spot inconsistencies or patterns of improper use.

For example, the system is configured to monitor and compare the quantities of particular drug flowing in the system from a manufacturer down to the patient. At any point in the distribution chain, when there is a spike in the quantity of a product either being shipped, stored or dispensed, that does not match the known quantity of authorized manufacture, and a signal may be generated indicating the entrance of a counterfeit product.

Conversely, when the amount of authorized sales is less than the known quantity of authorized manufacture of a product or that stored in a particular location, a signal may be generated indicating unauthorized diversion of a product. By monitoring the overall state of the supply chain, points of potential problems may be identified and investigated. Once potential counterfeiting or diversion is spotted, a signal may be generated indicating the entrance of a counterfeit product or an improper diversion.

Another problem a central database or control addresses is doctor shopping. Because the system receives patient prescription data, it is able to monitor and track the number of prescribers, by using for example DEA numbers, from which a particular patient obtains a particular subscription for a particular ailment. Once an irregularity is detected, a signal may be generated notifying past and future prescribers as well as other appropriate personnel.

Similarly, based again on patient prescription data, the system is also able to monitor and track the number of prescriptions a particular patient obtains for a certain drug. If the volume obtained is a mismatch with known treatment regimes, the system is designed to detect this irregularity. Again, a signal may be generated notifying past and future prescribers as well as other appropriate personnel that the individual may be obtaining excessive quantities of a drug.

In addition, by notifying the prescriber, the prescriber is able to compare the number of authorized prescriptions issued with the number detected. A mismatch may indicate potential forgeries.

In addition, prescriber information is obtained by the system and may be monitored, both at the issuing level and receipt by a dispenser. Again, an inconstancy may detect forged prescriptions.

Moreover, by monitoring the number of prescriptions issued to an individual for a particular drug, other patterns of misuse may be detected. These may include prescribers stepping outside the bounds of known treatment protocols.

In addition, the system is also designed to detect individual non-compliance instances such as forged prescriptions, counterfeit products and diversion as discussed in further detail below.

In one embodiment, labels are integral to a pill and one or more other levels of packaging. Other indicia or indicating devices that may be used include the pill itself or some other part of ingested medication. A patch or other device that is to be worn or carried by the person may also be used. Including the indicia or indicator in the pill or in close proximity to a patient, permits for patient scanning.

For example, should a person present to an ER or be pulled over for a violation, physicians, EMTs or police or DEA officials could scan the patient to determine what medications the patient had consumed. Further, if the identifying data contained in the ingested medication pills, or applied patches or other unit doses, were prescribed to another individual this would indicate diversion. By using the age RFID pedigree the source of diversion would be readily discoverable.

In another embodiment, the present invention provides that the bottle could be also be attached to or be integral with a transmitter which can transmit data each time a pill or unit dose is removed. This can be done by weight or by each pill or dose having an RFID label integral to it.

The use of the pills could be tracked by this manner which could be voluntary or involuntary by variation. The transmitter could be in the form of a credit card or other small device which would need to be presented to a pharmacy in order for the pharmacist to dispense medications, or to the prescriber and this may be linked to either the bottle or pills or both.

The data contained therein could be downloaded or otherwise transmitted at the time of the patient visit or telephonically or via Internet or other means in real time to the pharmacist, prescriber or others to keep track of the patient's use of the medication, and to the manufacturer who could keep the patient data in their database for the prevention of drug abuse.

For example, if the patient were to remove too many pills in too short a time, the physician, pharmacy or other individuals could be notified immediately. A mechanism could be incorporated such that any break in transmission or scheduled transmissions from the transmitter would also generate a signal notifying potential tampering.

In another embodiment, the present invention provides a method wherein each time a prescription is filled, the prescribing physician or entity, may be notified electronically by computer or other media. The dispensing entity could then verify that the patient is his, or this could be done automatically via comparison with a database. Should the prescription be unauthorized, for example, if the patient was not a patient of that prescribing entity or if the prescriber never wrote the prescription, or if the number of pills was forged, then the prescriber would be notified and depending upon preference, DEA or law enforcement will be notified as well. Local pharmacies or chain pharmacies may also be notified and computerized algorithms may be used to prevent further forgeries.

Next a pedigree would be generated for the pills that were illegally obtained and these could be traced by a passive or active scanning and identified much like a lost telephone using the RFID technology described above or by using Internet technology or by scanning through totals.

An important part of this invention involves computerized algorithms and Internet, telephonic or other electronic data collection and processing with comparisons to note irregularities and this could extend down to the end-user i.e. the patient from the point of import or manufacture.

1. A system for tracking one or more drugs comprising:
a central database adapted to track a drug through its lifecycle from point of manufacture, to distributor, to pharmacy, to one or more prescribers, and to use by a patient;
said system adapted to indicate unauthorized diversion of the drug:
said system adapted to recognize doctor shopping;
said system adapted to recognize inconsistencies or patterns indicating illicit or improper use of a drug;
said drug includes a plurality of pills, each of said pills having RFID tags;
said system further includes a RFID reader adapted to monitor the store and dispense said pills;
a container adapted to store said pills and said container is adapted to monitor the weight of said pills; and
said RFID reader sends a signal each time a pill is dispensed from the container to said central database.

2. The system of claim 1 wherein said inconsistency is an unaccounted increase in the quantity of a drug being shipped, stored or dispensed that does not match a predetermined quantity representing an authorized amount of manufacture.

3. The system of claim 2 wherein a signal is generated indicating the entrance of a counterfeit drug based on said inconsistency.

4. The system of claim 1 wherein said inconsistency is an unaccounted decrease in the quantity of a drug being shipped, stored or dispensed that does not match a predetermined quantity representing an authorized amount of manufacture.

5. The system of claim 4 wherein a signal is generated indicating that drugs have been diverted.

6. The system of claim 1 wherein said system is adapted to track the number of prescribers of a drug from which a patient obtains a prescription for a drug for a particular ailment and said system further adapted to compare the volume of drug obtained with a range of treatment regimens associated with the aliment.

7. The system of claim 6 wherein a signal is generated indicating that an excessive amount of a drug has been obtained when the amount of drug obtained is greater that a predetermined range of use.

8. The system of claim 7 wherein said signal is sent to a prescriber.

9. The system of claim 7 wherein said signal is sent to a dispenser.

10. The system of claim 1 wherein said system is adapted such that each time a prescription is filled, the prescriber is notified and said system is adapted to verify that the prescription was authorized by said prescriber.

11. The system of claim 10 wherein upon the detection of an unauthorized prescription, a signal is generated.

12. The system of claim 11 wherein said signal is sent to the prescriber.

13. (canceled)

14. (canceled)

15. (canceled)

16. The system of claim 1 wherein said system is adapted to track the number of pills dispensed and said system is further adapted to compare the volume of pills dispensed with a prescription stored in said central database.

17. (canceled)

18. The system of claim 1 wherein said changes in weight are correlated with the amount of pills dispensed.

19. The system of claim 18 wherein said system is adapted to track the number of pills dispensed and said system is further adapted to compare the volume of pills dispensed with a prescription stored in said central database.
